(19) United States
(12) Patent Application Publication
(10) Pub. No.: US 2025/0314610 A1
(43) Pub. Date: Oct. 9, 2025

(54) Taste Bud Cell Emulator Chip with Electrochemical Sensing, Cellular, and Neurological Processing

(71) Applicant: Georgia Tech Research Corporation, Atlanta, GA (US)

(72) Inventors: William D. Hunt, Atlanta, GA (US); Nael A. McCarty, Atlanta, GA (US); C. Shawn Dotson, Atlanta, GA (US); Andrew Eckford, Toronto (CA)

(21) Appl. No.: 19/170,975

(22) Filed: Apr. 4, 2025

Related U.S. Application Data
(60) Provisional application No. 63/574,793, filed on Apr. 4, 2024.

(51) Int. Cl. G01N 27/327; G01N 33/543; G01N 33/74

(52) U.S. Cl. CPC G01N 27/3276; G01N 33/54373; G01N 33/74; G01N 2333/726

(57) ABSTRACT

A taste bud emulator system comprising a plurality of circuits configured to interact with a sample and generate a plurality of output signals indicative of cellular responses of a taste bud cell to the sample. The plurality of output signals can comprise: an axon output signal indicative of an axonal output of a taste bud cell in response to interaction with the sample; a cell output signal indicative of a cell membrane voltage of a taste bud cell in response to interaction with the sample; and state space output signal indicative of a state of one or more cell membrane proteins of a taste bud cell in response to interaction with the sample.

### CROSS-REFERENCE TO RELATED APPLICATIONS

This application claims the benefit of U.S. Provisional Application Ser. No. 63/574,793, filed on 4 Apr. 2024, which is incorporated herein by reference in its entirety as if fully set forth below.

### FIELD OF THE DISCLOSURE

The present invention relates to the field of bioelectronic devices, specifically to a taste bud cell emulator chip that integrates electrochemical sensing with cellular and neurological processing to mimic the human taste perception mechanism.

### BACKGROUND

Taste perception is a complex process involving the interaction of taste molecules with taste receptor cells in the taste buds, followed by signal transduction to the brain. This process is essential for the detection of nutrients and harmful substances, influencing dietary choices and overall health. Traditional methods for studying taste perception include in vivo and in vitro assays, which are often time-consuming, expensive, and limited in their ability to replicate the full complexity of human taste sensation.

The assessment of the taste quality of a putative sweetener is made based on its relative similarity to sucrose (e.g., no “off” taste perceptions), fast onset of a sweet taste perception that does not linger, and sweet tastes that adapt or desensitizes the gustatory system.

The conundrum faced by companies in the food and beverage industry is this: they wish to provide no- or low-calorie products but not at the expense of taste and the biggest contributor to the caloric density of a beverage is added sugar. As such, companies are in a never-ending search for low- or no-calorie sweetener compounds that can help them to achieve this goal. The assessment of the taste quality of a putative sweetener is made based on its relative similarity to sucrose (e. g., no “off” taste perceptions), fast onset of a sweet taste perception that does not linger, and sweet tastes that adapt or desensitizes the gustatory system.

Recent advancements in bioelectronics and microfabrication have led to the development of various biosensors and lab-on-a-chip devices. These technologies have shown promise in mimicking biological processes and providing real-time analysis of biochemical interactions. However, existing devices for taste sensing are primarily focused on detecting specific taste molecules and lack the ability to emulate the integrated cellular and neurological processing involved in human taste perception. Thus, technological innovation is needed to provide systems and methods of multi-chip module that overcome the limitation of the conventional systems and method.

### BRIEF SUMMARY

An exemplary embodiment of the present disclosure provides a taste bud emulator system comprising a plurality of circuits configured to interact with a sample and generate a plurality of output signals indicative of cellular responses of a taste bud cell to the sample. The plurality of output signals can comprise: an axon output signal indicative of an axonal output of a taste bud cell in response to interaction with the sample; a cell output signal indicative of a cell membrane voltage of a taste bud cell in response to interaction with the sample; and state space output signal indicative of a state of one or more cell membrane proteins of a taste bud cell in response to interaction with the sample.

In any of the embodiments disclosed herein, the plurality of circuits can comprise a molecular recognition sensor circuit configured to interface with a sample and generate a unique ligand-receptor signal associated with the sample.

In any of the embodiments disclosed herein, the plurality of circuits can comprise a G-protein coupled receptor (GPCR) circuit configured to generate a GPCR output signal indicative of molecular recognition of the sample molecule. For example, if the receptor being utilized in the GPCR module is for sucrose, a similar, but not exact, signal will be generated when either glucose or fructose is presented as sucrose is made up of a combination of both glucose and fructose.

In any of the embodiments disclosed herein, the plurality of circuits can comprise a membrane channel electronics circuit configured to receive the unique ligand-receptor signal and the GPCR output signal and generate an MCE output signal indicative a response of one or more membrane channels to interaction with the sample.

In any of the embodiments disclosed herein, the plurality of circuits can comprise a cellular processor circuit configured to receive the MCE output signal and generate both an axon output signal indicative of an axonal output of a taste bud cell in response to interaction with the sample and a cell output signal indicative of a cell membrane voltage of a taste bud cell in response to interaction with the sample.

In any of the embodiments disclosed herein, the cell output signal can be indicative of a cell membrane voltage as a function of membrane location position and time of a taste bud cell in response to interaction with the sample.

In any of the embodiments disclosed herein, the plurality of circuits can comprise a protein state model circuit configured to receive the cell output signal and generate a state space output signal indicative of a state of one or more cell membrane proteins of a taste bud cell in response to interaction with the sample.

In any of the embodiments disclosed herein, the plurality of circuits can comprise at least one analog circuit and at least one digital circuit.

Another embodiment of the present disclosure provides a method of emulating a taste bud, the method comprising: providing a sample to be tested; interacting the sample with a plurality of circuits to generate a plurality of output signals indicative of cellular responses of a taste bud cell to the sample, the plurality of output signals comprising: an axon output signal indicative of an axonal output of a taste bud cell in response to interaction with the sample; a cell output signal indicative of a cell membrane voltage of a taste bud cell in response to interaction with the sample; and state space output signal indicative of a state of one or more cell membrane proteins of a taste bud cell in response to interaction with the sample.

In any of the embodiments disclosed herein, interacting the sample with a plurality of circuits can comprise generating a unique ligand-receptor signal associated with the sample.

In any of the embodiments disclosed herein, interacting the sample with a plurality of circuits can comprise generating a GPCR output signal indicative of molecular recognition of the sample molecule.

In any of the embodiments disclosed herein, interacting the sample with a plurality of circuits can comprise generating, based at least in part on the unique ligand-receptor signal and the GPCR output signal, an MCE output signal indicative a response of one or more membrane channels to interaction with the sample.

In any of the embodiments disclosed herein, interacting the sample with a plurality of circuits can comprise generating, based at least in part on the MCE output signal, an axon output signal indicative of an axonal output of a taste bud cell in response to interaction with the sample and a cell output signal indicative of a cell membrane voltage of a taste bud cell in response to interaction with the sample.

In any of the embodiments disclosed herein, the cell output signal cam be indicative of a cell membrane voltage as a function of membrane location position and time of a taste bud cell in response to interaction with the sample.

In any of the embodiments disclosed herein, interacting the sample with a plurality of circuits can comprise generating, based at least in part on the cell output signal, a state space output signal indicative of a state of one or more cell membrane proteins of a taste bud cell in response to interaction with the sample.

Another embodiments of present disclosure provides a method of determining a similarity of tastes of a first sample and a second sample, the method comprising: providing a taste bud emulator system as disclosed herein; interacting the first sample with a plurality of circuits of the taste bud emulator system to generate a first plurality of output signals based on the first sample; interacting the second sample with the plurality of circuits of the taste bud emulator system to generate a second plurality of output signals based on the second sample; and comparing the first plurality of output signals to the second plurality of output signals.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1 illustrates taste bud cell detection and signaling processes.

FIG. 2 illustrates a taste bud cell emulator system, in accordance with various embodiments of the present disclosure.

FIG. 3 illustrates an example illustration of a circuit for a cystic fibrosis transmembrane conductance regulator membrane channel, in accordance with various embodiments of the present disclosure.

FIG. 4 illustrates a portion of a taste bud cell emulator system comprising a molecular recognition circuit and a G-protein coupled receptor circuit, in accordance with various embodiments of the present disclosure.

FIG. 5 illustrates a portion of a taste bud cell emulator system comprising a molecular recognition circuit and a G-protein coupled receptor circuit, in accordance with various embodiments of the present disclosure.

FIG. 6 illustrates a portion of a taste bud cell emulator system comprising a membrane channel electronics circuit, in accordance with various embodiments of the present disclosure.

FIG. 7 illustrates a portion of a taste bud cell emulator system comprising a cellular processor circuit, in accordance with various embodiments of the present disclosure.

FIG. 8 illustrates a portion of a taste bud cell emulator system comprising a protein state model circuit, in accordance with various embodiments of the present disclosure

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

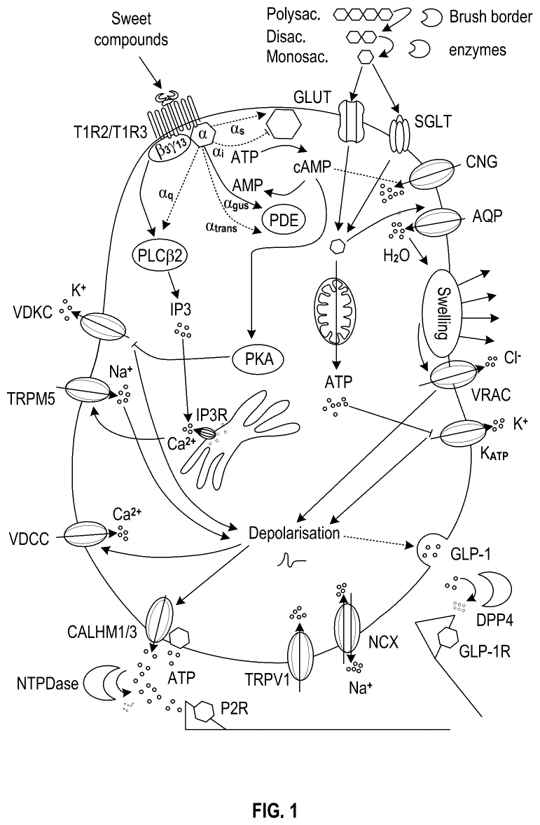

There has been quite a bit of work over the past twenty years or so, researching attempts to develop an electronic version of our sense of taste. A key distinguishing feature of certain embodiments of the present disclosure over those prior attempts, however, is the ability to some embodiments disclosed herein to mimic as closely as possible the taste bud cell. A remarkable amount of sensing, detection and signal processing takes place in taste bud cells as shown in FIG. 1. The action potentials, which are transmitted to the central nervous system (CNS) by the 7^{th}, 9^{th}, and 10^{th} cranial nerves, are the result of a great deal of local, in-cell processing. In the present disclosure, we will focus on the detection of sweet compounds such as fructose, glucose, sweeteners, etc., though the invention is not so limited; rather, as those skilled in the art would appreciate, embodiments of the present disclosure can be expandable to other classes of closely related molecules. These compounds bind individually to the ligand binding domains of the G-protein Coupled Receptor (GPCR), in this case a heterodimer of T1R2 and T1R3 proteins. This molecular recognition binding event sets off the cascade of events shown in FIG. 1 culminating in activation of neuronal signaling.

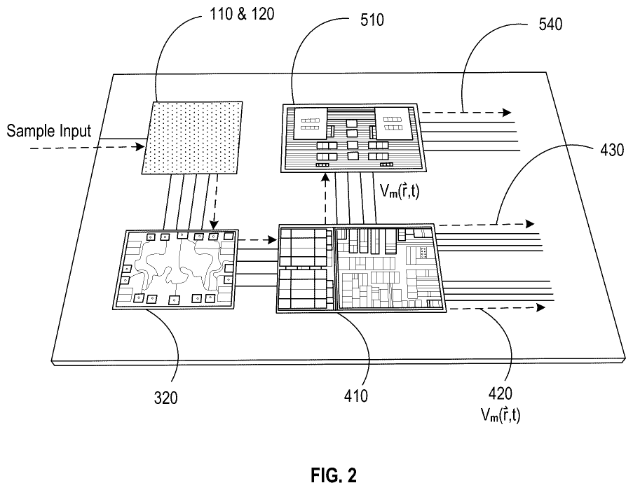

The essence of some embodiments of the present disclosure is that they mimic, in silico, this cellular process—almost at the molecular level-in a multi-chip module. As shown in FIG. 2, an exemplary embodiment of the present disclosure provides a TBC emulator system. The system can comprise a plurality of circuits configured to interact with a sample and generate a plurality of output signals indicative of cellular responses of a TBC to the sample. As used herein, term “circuit” is broadly defined to encompass any arrangement of interconnected electrical or electronic components designed to perform a specific function. This includes, but is not limited to, analog circuits, digital circuits, mixed-signal circuits, integrated circuits (ICs), and chips. A circuit may comprise various elements such as resistors, capacitors, inductors, diodes, transistors, and other semiconductor devices, as well as microcontrollers, microprocessors, and other programmable logic devices. The term also includes printed circuit boards (PCBs) and any other substrate or medium on which these components are mounted or embedded.

As shown in FIG. 2, the plurality of circuits can include a molecular recognition sensor circuit, a G-protein coupled receptor circuit, a membrane channel electronics circuit, a cellular processor circuit, and a protein state model circuit. Each of these circuits can be configured to perform a specific function in the emulation of a TBC, as will be discussed in more detail below. At a high level, the plurality of circuits can operate together to generate a plurality of output signals. In some embodiments, the plurality of output signals can include an axon output signal indicative of an axonal output of a taste bud cell in response to interaction with the sample. In some embodiments, the plurality of output signals can include a cell output signal indicative of a cell membrane voltage of a taste bud cell in response to interaction with the sample. In some embodiments, the plurality of output signals can include a state space output signal indicative of a state of one or more cell membrane proteins of a taste bud cell in response to interaction with the sample.

The molecular recognition sensor circuit (also referred to herein as a molecular recognition element can be biochemically configured to imitate the G-protein coupled receptor (GPCR) sweetness receptor. The process which takes place in the MRE and GPCR module/circuit represents the initiation of the cellular cascade which results in alterations in the taste bud cell (TBC) membrane voltage, Vₘ(r, t), and ultimately gives rise to the axonal output, which propagates on cranial nerves to the CNS.

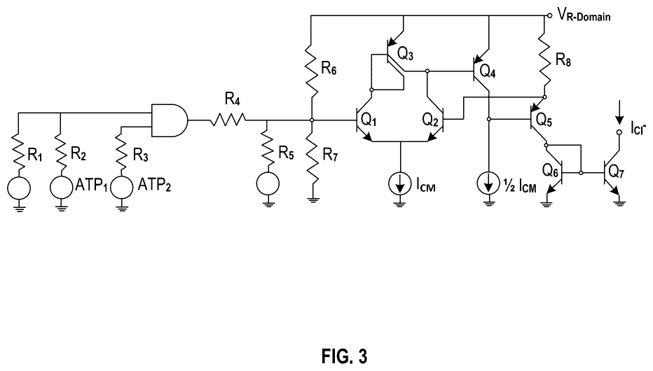

In some embodiments, the membrane channel electronics circuit (also referred to herein as a membrane channel electronics module) can comprise mixed analog/digital circuits that represent faithfully all of the membrane channels shown in FIG. 1. FIG. 3 provides a transistor level circuit developed for the Cystic Fibrosis Transmembrane Conductance Regulator (CFTR) membrane channel. The simulation results from this model match precisely patch clamp measurements made of a single CFTR membrane channel. This is the general approach to all features of the multi-chip module—molecular level mimicry.

The cellular processor circuit (also referred to herein as a cellular processor module) can be an embedded system which embodies in software the known cellular processes which result in a modification of the cell membrane voltage, Vm, as a function of membrane location position and time. In addition, in some embodiments, the cellular processor circuit can compute and generates the axonal output signal.

The protein state model circuit (also referred to herein as a protein state model) can also be an embedded system and can compute, via the stochastic techniques we have developed (see A. S. Moffett et al., “Permissive and nonpermissive channel closings in CFTR revealed by a factor graph inference algorithm,” Biophysical Reports, vol. 2, no. 4, 2022 [16], the state of relevant membrane proteins). The technique is a methodology to extract from the membrane channel currents, the states of those membrane proteins. This technique can be used to test and validate the output of the chip in terms of its biophysical realism, compared with the actual biological system.

In summary, the TBC emulators disclosed herein can provides one with both the axon output resulting from the introduction of a tastant (sample) and all of the information that the TBC had when it generated the axonal output. For example, in some embodiments the TBC emulator system can include a mimic of the cellular processing and membrane channel electronics that lead to axonal output, the cellular output and the state space representation of membrane channel response.

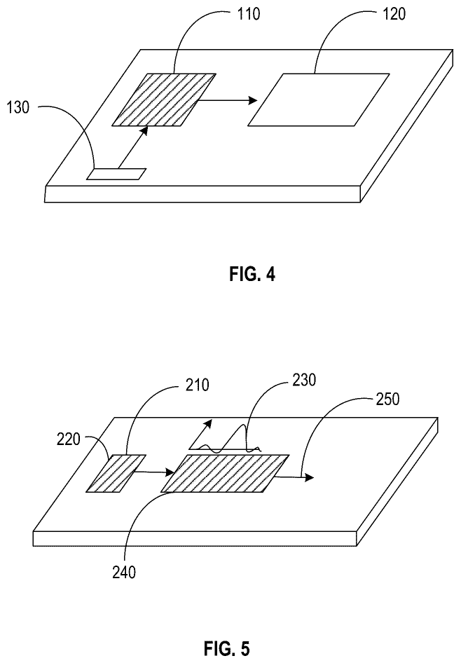

FIG. 4 provides an exemplary front end of the TBC Emulator. The molecular recognition sensing circuit can comprise a specially designed biolayer that is primarily, and almost exclusively, responsive to molecules which possess a sugar moiety (though as discussed above, other moieties can be recognized in other embodiments). In some embodiments, this layer can be immobilized onto a metal layer, for example gold, via an alkane thiol immobilization protocol, into a self-assembled monolayer. In some embodiments, other silane chemistries could be utilized to immobilize the molecular recognition sensing circuit onto a dielectric such as silicon nitride.

In some embodiments, the molecular recognition sensing circuit can comprise a multi-state protein designed to exhibit various conformational changes associated with various binding events (see M. F. Sauer, A. M. Sevy, J. E. Crowe Jr, and J. Meiler, “Multi-state design of flexible proteins predicts sequences optimal for conformational change,” PLOS Computational Biology, vol. 16, no. 2, p. e1007339, 2020). In some embodiments, the molecular recognition sensing circuit could be constructed, at least in part, from aptamers which are short sequences of artificial DNA, RNA, XNA or peptides that bind a specific target molecule, such as a sugar tastant. In some embodiments, the molecular recognition sensing circuit can comprise a recombinant protein which is derived from heterologous expression of the gene encoding the GPCR in the taste bud cell.

As shown in FIG. 4, in some embodiments, the molecular recognition sensing circuit 110 can be addressed by two different circuits. One is a mimic of the CFTR membrane channel 130 (see W. D. Hunt et al., “A transistor model for the cystic fibrosis transmembrane conductance regulator,” Biophysical Reports, vol. 3, no. 2, 2023). From these emulated patch clamp results, we can assess the state of the molecular recognition sensing circuit 110 by a technique we have previously reported (see A. S. Moffett et al., “Permissive and nonpermissive channel closings in CFTR revealed by a factor graph inference algorithm,” Biophysical Reports, vol. 2, no. 4, 2022). In accordance with various embodiments of the present disclosure, the design of the molecular recognition sensing circuit can be iterated over a variety of tastants so as to refine the molecular recognition sensing circuit itself in its ability to assess whether the recognition pocket is close to the optimal receptor for various sweeteners.

In addition, the molecular recognition sensing circuit can be addressed electrochemically by a GPCR emulator circuit 120. In the TBC, the binding of a tastant/ligand to the receptor as shown in FIG. 1 generates a cascade of events that essentially represent an effective amplification. An analogy would be a photomultiplier tube or avalanche photodiode wherein a single incident photon generates perhaps one hundred electrons as an output.

FIG. 5 provides more of the details of an exemplary molecular recognition sensing circuit 210, which can comprise a biosensor, and the GPCR emulator circuit 240. To illustrate—a sample tastant 220, in this particular case a sugar molecule-binds to the receptor in the molecular recognition sensing circuit 210 and this molecular binding interaction is transcribed into a ligand—receptor signal 230 which can be considered a signature associated with the tastant—somewhat analogous to a radar signature. Accordingly, the molecular recognition sensing circuit 210 can be configured to interface with a sample and generate a unique ligand-receptor signal associated with the sample. This signature can then go to the GPCR Emulator circuit 240 and drives the cascade of cellular events which would result within the TBC. In some embodiments, the GPCR Emulator circuit 240 can be an adaptive embedded system. Accordingly, the GPCR Emulator circuit can be adaptive and can evolve as more is discovered about the GPCR role in the function of the TBC. The GPCR circuit output 250 can then drive a number of cellular components including the membrane channels as is shown in FIG. 1.

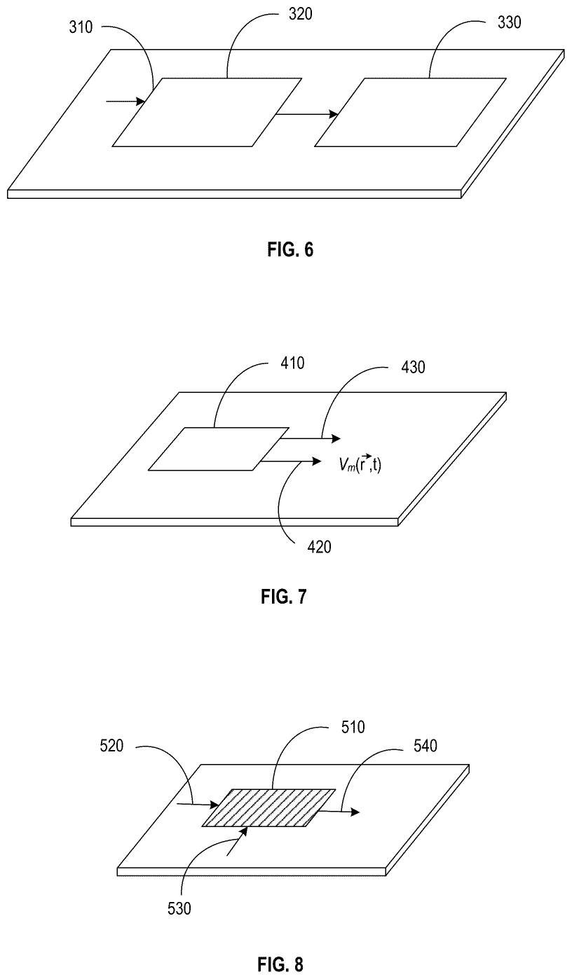

FIG. 6 illustrates the impact that the GPCR circuit output 250 and 310 has on the myriad of membrane channels. The membrane channel electronics circuit 320 can comprise individual mixed analog/digital circuits such as the one presented in FIG. 2. The membrane channel electronics circuit 320 can include mixed analog/digital circuits representing the known channels as shown in FIG. 1. The function of these circuits can be to emulate the conversion of GPCR output to ion current flow and membrane voltage, Vₘ. The cellular processor circuit 330 can be a programmable embedded processor which takes as inputs membrane channel ion currents as well as other intracellular processes resulting from the GPCR output. Accordingly, in some embodiments, the membrane channel electronics circuit can be configured to receive the unique ligand-receptor signal and the GPCR output signal and generate an MCE output signal indicative a response of one or more membrane channels to interaction with the sample.

FIG. 7 provides an exemplary cellular processor circuit and its outputs. The cellular processor circuit 410 too can comprise an embedded system and generate a cell output signal 420 which can be the membrane voltage, Vₘ(\vec{r}, t) as a function of time and position around the perimeter of the cell. In addition, in some embodiments, this embedded system can compute the axon output 430 which mimics the actual axonal output generated by the TBC. Accordingly, in some embodiments, the cellular processor circuit can be configured to receive the MCE output signal and generate (1) an axon output signal indicative of an axonal output of a taste bud cell in response to interaction with the sample and (2) a cell output signal indicative of a cell membrane voltage of a taste bud cell in response to interaction with the sample.

FIG. 8 provides an exemplary protein state model circuit 510. In some embodiments, the protein state model circuit 510 can be an embedded system that takes as its input the membrane channel outputs 520 and the membrane voltage outputs 530 and determines the state of various important proteins in the detection process, including the MRE. Accordingly, in some embodiments, the protein state model circuit 510 can be configured to receive the cell output signal and generate a state space output signal indicative of a state of one or more cell membrane proteins of a taste bud cell in response to interaction with the sample.

In addition to the TBC emulator systems discussed above, the present disclosure also provides methods of emulating a TBC. An exemplary method comprises: providing a sample to be tested; interacting the sample with a plurality of circuits to generate a plurality of output signals indicative of cellular responses of a taste bud cell to the sample, the plurality of output signals comprising: an axon output signal indicative of an axonal output of a taste bud cell in response to interaction with the sample; a cell output signal indicative of a cell membrane voltage of a taste bud cell in response to interaction with the sample; and state space output signal indicative of a state of one or more cell membrane proteins of a taste bud cell in response to interaction with the sample.

In some embodiments, interacting the sample with a plurality of circuits can comprise generating a unique ligand-receptor signal associated with the sample.

In some embodiments, interacting the sample with a plurality of circuits can comprise generating a GPCR output signal indicative of molecular recognition of the sample molecule. For example, if the receptor being utilized in the GPCR module is for sucrose, a similar, but not exact, signal will be generated when either glucose or fructose is presented as sucrose is made up of a combination of both glucose and fructose.

In some embodiments, interacting the sample with a plurality of circuits can comprise generating, based at least in part on the unique ligand-receptor signal and the GPCR output signal, an MCE output signal indicative a response of one or more membrane channels to interaction with the sample.

In some embodiments, interacting the sample with a plurality of circuits can comprise generating, based at least in part on the MCE output signal, an axon output signal indicative of an axonal output of a taste bud cell in response to interaction with the sample and a cell output signal indicative of a cell membrane voltage of a taste bud cell in response to interaction with the sample.

In some embodiments, interacting the sample with a plurality of circuits can comprise generating, based at least in part on the cell output signal, a state space output signal indicative of a state of one or more cell membrane proteins of a taste bud cell in response to interaction with the sample

In addition to TBC emulator system and methods, the present disclosure also provides methods of determining a similarity of tastes of a first sample and a second sample using the TBC emulator systems disclosed herein. In an exemplary embodiments, the method comprises: providing a TBC emulator system; interacting the first sample with the plurality of circuits of the TBC emulator system to generate a first plurality of output signals based on the first sample; interacting the second sample with the plurality of circuits of the TBC emulator system to generate a second plurality of output signals based on the second sample; and comparing the first plurality of output signals to the second plurality of output signals. The comparison can provide an indication on how comparable the taste of the first sample to the taste of the second sample may be to a person.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

What is claimed is:

1. A taste bud emulator system comprising:
a plurality of circuits configured to interact with a sample and generate a plurality of output signals indicative of cellular responses of a taste bud cell to the sample, the plurality of output signals comprising:
an axon output signal indicative of an axonal output of a taste bud cell in response to interaction with the sample;
a cell output signal indicative of a cell membrane voltage of a taste bud cell in response to interaction with the sample; and
state space output signal indicative of a state of one or more cell membrane proteins of a taste bud cell in response to interaction with the sample.

2. The taste bud emulator system of claim 1, wherein the plurality of circuits comprises a molecular recognition sensor circuit configured to interface with a sample and generate a unique ligand-receptor signal associated with the sample.

3. The taste bud emulator system of claim 2, wherein the plurality of circuits comprises a G-protein coupled receptor circuit configured to generate a GPCR output signal indicative of molecular recognition of the sample molecule.

4. The taste bud emulator system of claim 3, wherein the plurality of circuits comprises a membrane channel electronics circuit configured to receive the unique ligand-receptor signal and the GPCR output signal and generate an MCE output signal indicative a response of one or more membrane channels to interaction with the sample.

5. The taste bud emulator system of claim 4, wherein the plurality of circuits comprises a cellular processor circuit configured to receive the MCE output signal and generate an axon output signal indicative of an axonal output of a taste bud cell in response to interaction with the sample and a cell output signal indicative of a cell membrane voltage of a taste bud cell in response to interaction with the sample.

6. The taste bud emulator system of claim 5, wherein cell output signal is indicative of a cell membrane voltage as a function of membrane location position and time of a taste bud cell in response to interaction with the sample.

7. The taste bud emulator system of claim 5, wherein the plurality of circuits comprises a protein state model circuit configured to receive the cell output signal and generate a state space output signal indicative of a state of one or more cell membrane proteins of a taste bud cell in response to interaction with the sample.

8. The taste bud emulator system of claim 1, wherein the plurality of circuits comprise at least one analog circuit and at least one digital circuit.

9. A method of emulating a taste bud, the method comprising:
providing a sample to be tested;
interacting the sample with a plurality of circuits to generate a plurality of output signals indicative of cellular responses of a taste bud cell to the sample, the plurality of output signals comprising:
an axon output signal indicative of an axonal output of a taste bud cell in response to interaction with the sample;
a cell output signal indicative of a cell membrane voltage of a taste bud cell in response to interaction with the sample; and
state space output signal indicative of a state of one or more cell membrane proteins of a taste bud cell in response to interaction with the sample.

10. The method of claim 9, wherein interacting the sample with a plurality of circuits comprises generating a unique ligand-receptor signal associated with the sample.

11. The method of claim 10, wherein interacting the sample with a plurality of circuits comprises generating a GPCR output signal indicative of molecular recognition of the sample molecule.

12. The method of claim 11, wherein interacting the sample with a plurality of circuits comprises generating, based at least in part on the unique ligand-receptor signal and the GPCR output signal, an MCE output signal indicative a response of one or more membrane channels to interaction with the sample.

13. The method of claim 12, wherein interacting the sample with a plurality of circuits comprises generating, based at least in part on the MCE output signal, an axon output signal indicative of an axonal output of a taste bud cell in response to interaction with the sample and a cell output signal indicative of a cell membrane voltage of a taste bud cell in response to interaction with the sample.

14. The method of claim 13, wherein the cell output signal is indicative of a cell membrane voltage as a function of membrane location position and time of a taste bud cell in response to interaction with the sample.

15. The method of claim 13, wherein interacting the sample with a plurality of circuits comprises generating, based at least in part on the cell output signal, a state space output signal indicative of a state of one or more cell membrane proteins of a taste bud cell in response to interaction with the sample.

16. The method of claim 9, wherein the plurality of circuits comprise at least one analog circuit and at least one digital circuit.

17. A method of determining a similarity of tastes of a first sample and a second sample, the method comprising:
providing the taste bud emulator system of claim 1;
interacting the first sample with the plurality of circuits to generate a first plurality of output signals based on the first sample;
interacting the second sample with the plurality of circuits to generate a second plurality of output signals based on the second sample; and
comparing the first plurality of output signals to the second plurality of output signals.

18. The method of claim 17, wherein the taste bud emulator system is the taste bud emulator system of claim 7.
